(19) United States
(12) Patent Application Publication
(10) Pub. No.: US 2019/0043607 A1
(43) Pub. Date: Feb. 7, 2019

(54) DENTAL CHARTING DATA PROCESSING SYSTEMS AND RELATED METHODS

(71) Applicant: Orista, Inc., Suwanee, GA (US)

(72) Inventors: Walter Sears, Suwanee, GA (US); George MacMaster, Suwanee, GA (US)

(21) Appl. No.: 16/056,055

(22) Filed: Aug. 6, 2018

Related U.S. Application Data
(60) Provisional application No. 62/541,507, filed on Aug. 4, 2017.

(51) Int. Cl. G16H 10/60; G16H 15/00; G16H 30/20; G06F 3/0482

(52) U.S. Cl. CPC G16H 10/60; G06F 3/0482; G16H 30/20; G16H 15/00

(57) ABSTRACT

A dental charting data processing system and method enable a user to enter charting data during a dental examination by: (1) identifying a particular tooth; (2) identifying a particular surface of the particular tooth; (3) making a particular observation regarding the particular tooth; (4) making a treatment recommendation; and (4) repeating this process for each of the patient's teeth, without leaving a single graphical user interface. After the examination, a plain-language patient report is created using stored language associated with the input provided to the interface during the examination.

### CROSS REFERENCE TO RELATED APPLICATIONS

This application claims priority from U.S. Provisional Patent Application Ser. No. 62/541,507, filed Aug. 4, 2017. The disclosure of which is hereby incorporated herein by reference in its entirety.

### BACKGROUND

Individuals seeking dental care often have the records of their examinations entered into a chart. The completed charts may exhibit the locations of caries (e.g., cavities), broken and missing teeth, etc. Any work previously performed by a dentist, including restorations, crowns and bridges, may also be displayed. Charting during an examination can prove difficult without the aid of a knowledgeable assistant, and summarizing information in a way that patients can understand may be time-consuming. Moreover, charting by entering free form text for every entry is time consuming and reduces the number of patients that can be seen in a work day. Accordingly, there is a need for improved systems and methods that address these and other needs.

### SUMMARY

It should be appreciated that this Summary is provided to introduce a selection of concepts in a simplified form that are further described below in the Detailed Description. This Summary is not intended to be used to limit the scope of the claimed subject matter.

According to a first aspect of the disclosure, a computer-implemented data processing method is provided for electronically charting dental information for an individual. The method includes creating an electronic record for the individual and digitally storing the record. A graphical user interface is provided for charting dental information for each tooth associated with the individual, the graphical user interface includes inputs corresponding to the individual's teeth. A number of observation inputs are provided, each of which is associated with a particular observation. A number of treatment inputs are provided, each of which is associated with a particular treatment. Input data is electronically received. The input data includes a first input including a selection of a particular tooth of the individual's teeth and a second input associated with the first input, the second input including at least one observation or at least one treatment. The particular tooth is electronically associated with the observation or the treatment, and the input data is electronically associated with the electronic record for the individual. A summary report is generated of the at least one observation or the at least one treatment for the particular tooth, including a plain-language description of the at least one observation or the at least one treatment and a diagrammatic representation of the particular tooth relative to the plurality of teeth.

According to another aspect, a computer-readable storage medium storing computer-executable instructions is provided. According to the computer-executable instructions, a graphical user interface is provided for charting dental information for each tooth associated with the individual. The graphical user interface includes a number of inputs corresponding to the individual's teeth, a number of observation inputs associated with particular observations, and a number of treatment inputs associated with particular treatments. Input data is electronically received, including a first input corresponding to a selection of a particular tooth of the plurality of teeth, and a second input associated with the first input. The second input includes at least one observation or at least one treatment. A graphical representation is provided on the graphical user interface of the particular tooth associated with the at least one first input, the graphical representation including a representation of the at least one observation or the at least one treatment corresponding to the second input.

According to yet another aspect, a computer-implemented data processing method is provided for electronically charting dental information for an individual. The method includes accessing an electronic record for the individual and providing a number of graphical user interfaces corresponding to the electronic record associated with the individual and one or more dental examinations or treatments associated with the individual. Input is received via one or more of the graphical user interfaces and the electronic record for the individual is digitally updated according to the input. A first graphical user interface includes a number of inputs corresponding to the individual's teeth, a number of observation inputs associated with particular observations, and a number of treatment inputs associated with particular treatments.

The features, functions, and advantages that have been discussed can be achieved independently in various embodiments of the present disclosure or may be combined in yet other embodiments, further details of which can be seen with reference to the following description and drawings.

### BRIEF DESCRIPTION OF THE DRAWINGS

Various embodiments of a Dental Charting Data Processing System and Method are described below. In the course of this description, reference will be made to the accompanying drawings, which are not necessarily drawn to scale and wherein:

FIG. 1 depicts a Dental Charting and Data Management System according to particular embodiments.

FIG. 2 is a schematic diagram of a computer (such as the Dental Data Management Server 110, or one or more remote computing devices 130) that is suitable for use in various embodiments of the Dental Charting and Data Management System shown in FIG. 1.

FIG. 3 depicts an exemplary flow chart of operations that make up a Dental Charting and Data Management Module 300, according to particular embodiments, which the Dental Charting and Data Management System may be configured to execute.

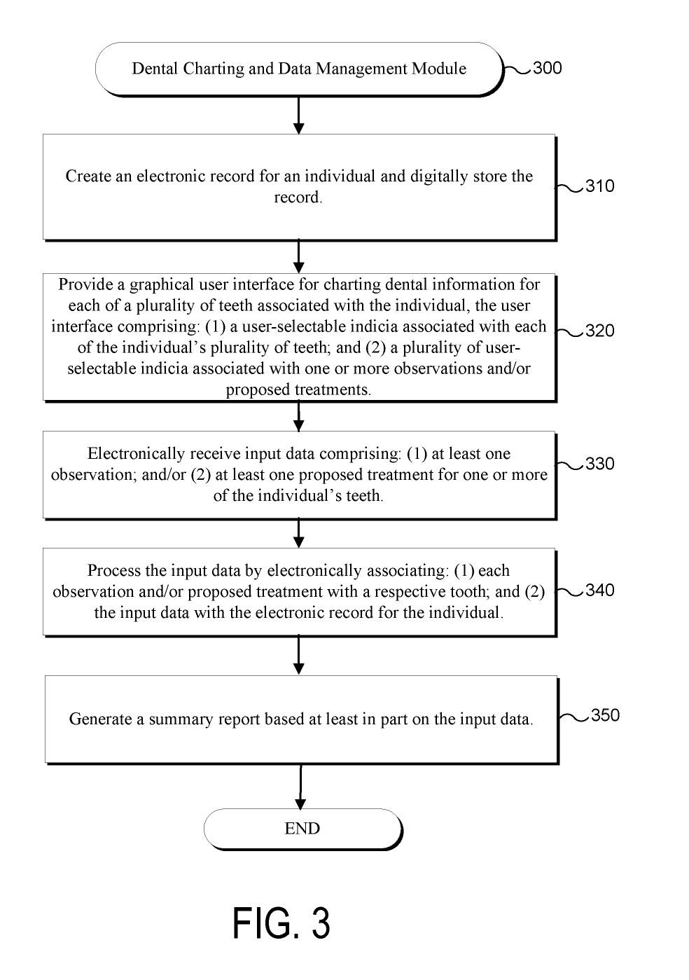

FIG. 4 depicts an exemplary snap shot interface according to various embodiments.

FIG. 5 depicts an exemplary health history summary interface according to various embodiments.

FIG. 6 depicts an exemplary dental charting interface according to various embodiments.

FIG. 7 depicts an exemplary caries risk interface according to various embodiments.

FIG. 8 depicts an exemplary ortho-tracking interface according to various embodiments.

FIG. 9 depicts an exemplary hygiene interface according to various embodiments.

FIG. 10 depicts an exemplary narrative interface according to various embodiments.

FIG. 11 depicts an exemplary exam and treatment plan interface according to various embodiments.

FIG. 12 depicts an exemplary patient report interface according to various embodiments.

FIG. 13 depicts an exemplary family history interface according to various embodiments.

FIG. 14 depicts an exemplary operative treatment interface according to various embodiments.

FIG. 15 depicts an exemplary post-examination treatment interface according to various embodiments.

FIG. 16 depicts an exemplary procedures interface according to various embodiments.

FIG. 17 depicts an exemplary receipt interface according to various embodiments.

### DETAILED DESCRIPTION OF VARIOUS EMBODIMENTS

Various embodiments will now be described in greater detail. It should be understood that the invention may be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

### Exemplary Technical Platforms

As will be appreciated by one skilled in the relevant field, the present invention may be, for example, embodied as a computer system, a method, or a computer program product. Accordingly, various embodiments may take the form of an entirely hardware embodiment, an entirely software embodiment, or an embodiment combining software and hardware aspects. Furthermore, particular embodiments may take the form of a computer program product stored on a computer-readable storage medium having computer-readable instructions (e.g., software) embodied in the storage medium. Various embodiments may take the form of web-implemented computer software. Any suitable computer-readable storage medium may be utilized including, for example, hard disks, compact disks, DVDs, optical storage devices, and/or magnetic storage devices.

Various embodiments are described below with reference to block diagrams and flowchart illustrations of methods, apparatuses (e.g., systems), and computer program products. It should be understood that each block of the block diagrams and flowchart illustrations, and combinations of blocks in the block diagrams and flowchart illustrations, respectively, can be implemented by a computer executing computer program instructions. These computer program instructions may be loaded onto a general purpose computer, special purpose computer, or other programmable data processing apparatus to produce a machine, such that the instructions which execute on the computer or other programmable data processing apparatus to create means for implementing the functions specified in the flowchart block or blocks.

These computer program instructions may also be stored in a computer-readable memory that can direct a computer or other programmable data processing apparatus to function in a particular manner such that the instructions stored in the computer-readable memory produce an article of manufacture that is configured for implementing the function specified in the flowchart block or blocks. The computer program instructions may also be loaded onto a computer or other programmable data processing apparatus to cause a series of operational steps to be performed on the computer or other programmable apparatus to produce a computer implemented process such that the instructions that execute on the computer or other programmable apparatus provide steps for implementing the functions specified in the flowchart block or blocks.

Accordingly, blocks of the block diagrams and flowchart illustrations support combinations of mechanisms for performing the specified functions, combinations of steps for performing the specified functions, and program instructions for performing the specified functions. It should also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and other hardware executing appropriate computer instructions.

### Example System Architecture

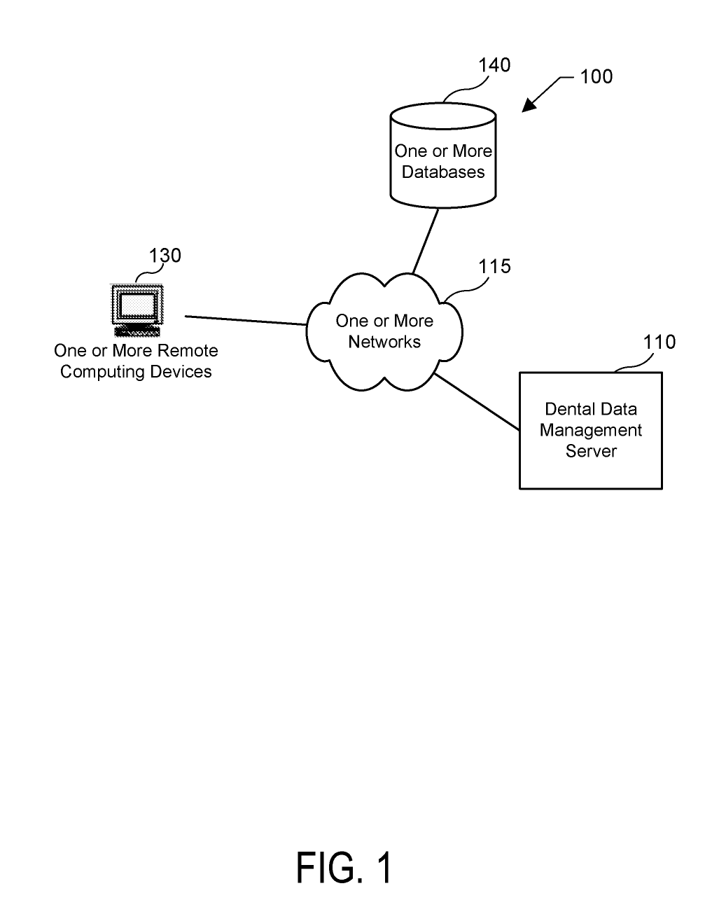

FIG. 1 is a block diagram of a Dental Charting and Data Management System 100 according to a particular embodiment. In some embodiments, the Dental Charting and Data Management System 100 is configured to provide a platform for managing entry, organization, and presentation of dental charting data. The system may, for example, be implemented in the context of a dental practice and be configured to: (1) enable simple, straightforward entry of data related to a dental examination such as observations, treatments plans, etc. for particular teeth; (2) process entered data; and (3) present the data for consumption by one or more individuals (e.g., patients, parents of young patients, etc.).

As may be understood from FIG. 1, the Dental Charting and Data Management System 100 includes one or more computer networks 115, a Dental Data Management Server 110, one or more remote computing devices 130 (e.g., such as a desktop computer, laptop computer, tablet computer, smartphone, etc.), and One or More Databases 140. In particular embodiments, the one or more computer networks 115 facilitate communication between the Dental Data Management Server 110, one or more remote computing devices 130 (e.g., a desktop computer, laptop computer, tablet computer, etc.), and one or more databases 140.

The one or more computer networks 115 may include any of a variety of types of wired or wireless computer networks such as the Internet, a private intranet, a public switch telephone network (PSTN), or any other type of network. The communication link between the Dental Data Management Server 110 and Database 140 may be, for example, implemented via a Local Area Network (LAN) or via the Internet.

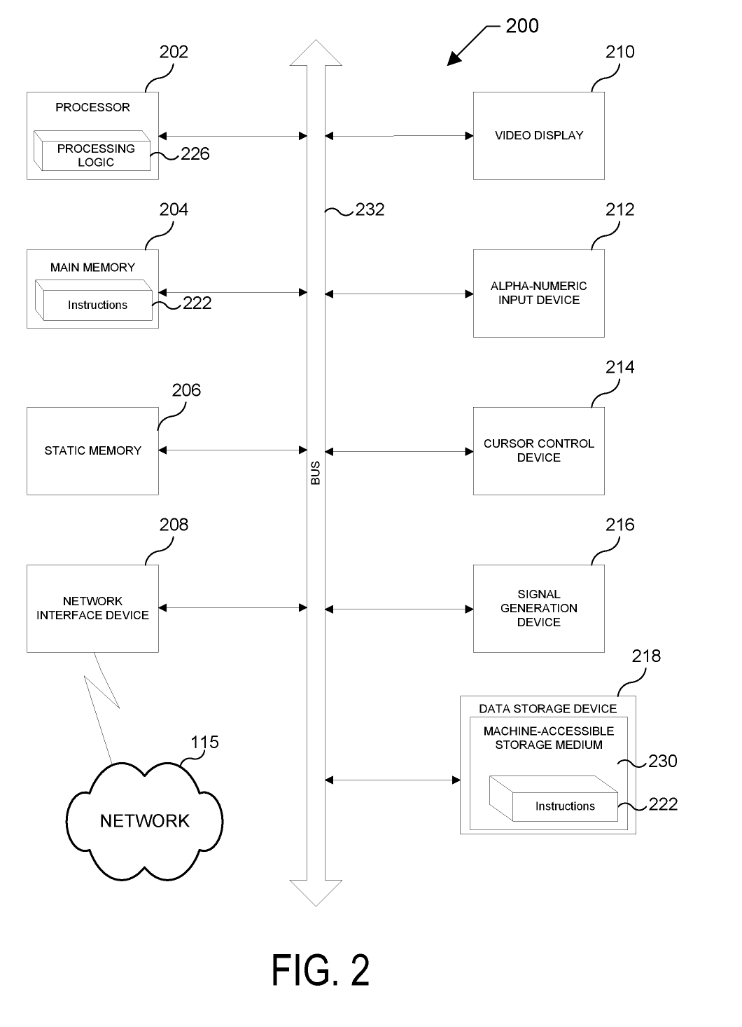

FIG. 2 illustrates a diagrammatic representation of a computer 200 that can be used within the Dental Charting and Data Management System 100, for example, as a client computer (e.g., one or more remote computing devices 130 shown in FIG. 1), or as a server computer (e.g., Dental Data Management Server 110 shown in FIG. 1). In particular embodiments, the computer 200 may be suitable for use as a computer within the context of the Dental Charting and Data Management System 100 that is configured to receive data input, generate reports, etc.

In particular embodiments, the computer 200 may be connected (e.g., networked) to other computers in a LAN, an intranet, an extranet, and/or the Internet. As noted above, the computer 200 may operate in the capacity of a server or a client computer in a client-server network environment, or as a peer computer in a peer-to-peer (or distributed) network environment. The Computer 200 may be a personal computer (PC), a tablet PC, a set-top box (STB), a Personal Digital Assistant (PDA), a cellular telephone, a web appliance, a server, a network router, a switch or bridge, or any other computer capable of executing a set of instructions (sequential or otherwise) that specify actions to be taken by that computer. Further, while only a single computer is illustrated, the term “computer” shall also be taken to include any collection of computers that individually or jointly execute a set (or multiple sets) of instructions to perform any one or more of the methodologies discussed herein.

An exemplary computer 200 includes a processing device 202, a main memory 204 (e.g., read-only memory (ROM), flash memory, dynamic random access memory (DRAM) such as synchronous DRAM (SDRAM) or Rambus DRAM (RDRAM), etc.), static memory 206 (e.g., flash memory, static random access memory (SRAM), etc.), and a data storage device 218, which communicate with each other via a bus 232.

The processing device 202 represents one or more general-purpose processing devices such as a microprocessor, a central processing unit, or the like. More particularly, the processing device 202 may be a complex instruction set computing (CISC) microprocessor, reduced instruction set computing (RISC) microprocessor, very long instruction word (VLIW) microprocessor, or processor implementing other instruction sets, or processors implementing a combination of instruction sets. The processing device 202 may also be one or more special-purpose processing devices such as an application specific integrated circuit (ASIC), a field programmable gate array (FPGA), a digital signal processor (DSP), network processor, or the like. The processing device 202 may be configured to execute processing logic 226 for performing various operations and steps discussed herein.

The computer 200 may further include a network interface device 208. The computer 200 also may include a video display unit 210 (e.g., a liquid crystal display (LCD) or a cathode ray tube (CRT)), an alphanumeric input device 212 (e.g., a keyboard), a cursor control device 214 (e.g., a mouse), and a signal generation device 216 (e.g., a speaker).

The data storage device 218 may include a non-transitory computer-accessible storage medium 230 (also known as a non-transitory computer-readable storage medium or a non-transitory computer-readable medium) on which is stored one or more sets of instructions (e.g., software instructions 222) embodying any one or more of the methodologies or functions described herein. The software instructions 222 may also reside, completely or at least partially, within main memory 204 and/or within processing device 202 during execution thereof by computer 200—main memory 204 and processing device 202 also constituting computer-accessible storage media. The software instructions 222 may further be transmitted or received over a network 115 via network interface device 208.

While the computer-accessible storage medium 230 is shown in an exemplary embodiment to be a single medium, the term “computer-accessible storage medium” should be understood to include a single medium or multiple media (e.g., a centralized or distributed database, and/or associated caches and servers) that store the one or more sets of instructions. The term “computer-accessible storage medium” should also be understood to include any medium that is capable of storing, encoding or carrying a set of instructions for execution by the computer and that cause the computer to perform any one or more of the methodologies of the present invention. The term “computer-accessible storage medium” should accordingly be understood to include, but not be limited to, solid-state memories, optical and magnetic media, etc.

### Overview

In particular embodiments, a Dental Charting and Data Management System 100 is configured to enable a user to enter charting data during a dental examination without the user needing to have any knowledge of dental or technical medical terms. When performing a dental examination on a patient, a dentist may, for example, enlist the aid of an assistant to enter the dentist's observations, diagnoses, proposed treatments, etc. into the patient's chart. The dentist may, for example: (1) identify a particular tooth (e.g., by number, letter, name, etc.); (2) identify a particular surface of the particular tooth (e.g., distal, occlusal, buccal, mesial, lingual, palatial, etc.); (3) make a particular observation regarding the particular tooth (e.g., erupting, dark, etc.); and (4) repeat this process for each of the patient's teeth.

As the dentist is performing the examination of the patient, the assistant may be required to ensure that observations and treatments the dentist is noting are being charted accurately and quickly. This may, for example, require the assistant to be familiar with and have knowledge of, the various terms used by the dentist to identify the particular teeth and their respective surfaces. In particular embodiments, the Dental Charting and Data Management System 100 is configured to enable a user to enter charting information for the dentist during an examination without having any knowledge of the technical language being utilized or knowledge of the tooth surfaces or location. In various embodiments, the examination may be based on, for example, visual inspection, mechanical inspection, radiograph inspection, or other suitable technique. Because a dentist can see a large number of patients during a single day (e.g., exceeding eighty patients), it is important for a dental practice to ensure that examinations are properly charted and recorded while also ensuring that the dentist has sufficient time to complete his or her examinations.

In various embodiments, the system is configured to provide a graphical user interface comprising a plurality of user-selectable inputs. These user-selectable inputs may comprise, for example: (1) a user-selectable input for each of a plurality of an individual's teeth (e.g., including both permanent teeth, primary teeth, third molars, etc.); (2) a plurality of user-selectable inputs associated with various observations; and (3) a plurality of user-selectable inputs associated with proposed treatments. In particular embodiments, the system is configured to display all of the user-selectable inputs on a single, straightforward user interface screen. In various embodiments, the user interface is configured to enable simple entry of charting information. In some embodiments, for example, the user interface does not utilize drop down menus or other bulky menus that the user must navigate to select particular data for entry. In such embodiments, the selectable data for entry is contained in a single, easy to navigate display screen.

In particular embodiments, while the user is entering data for the dentist during the examination, the user: (1) selects a first user-selectable input (e.g., indicia) associated with a first tooth; and (2) selects one or more user-selectable inputs associated with one or more observations made or proposed treatments stated by the dentist. The system may then be configured to associate the selected observations and/or treatment(s) with the first tooth and store that association in memory. The system may, for example, generate or update a data structure or database associated with the patient to include the selected observation or proposed treatment for the first tooth. In various embodiments, the selected one or more observations and/or one or more treatments have a predefined narrative associated with the selection so that the predefined narrative can be entered into the patient's dental record without the dentist or assistant having to manually enter the narrative into the system. In various embodiments, the system may allow the dentist or the assistant to edit or update the predefined narrative when necessary.

The user may then repeat this process for each of the patient's remaining teeth. In various embodiments, the system is configured to store the observation and treatment data for each tooth in an electronic record (e.g., database) associated with the patient. In various embodiments, the dentist may provide his or her observations in the form of an abbreviation or acronym (e.g., such as any suitable dental practice abbreviation or dental practice abbreviations from the American Dental Association). The abbreviation ‘DO’ may, for example, refer to distal occlusal. When making his observations, the dentist may simply recite “tooth number 5, DO.” The user may then select tooth number 5 on the user interface and then select the DO labeled user-selectable menu item. As may be understood in light of this disclosure, the user entering the charting information, when using the system, would not be required to know which particular tooth is tooth five, or what DO means. The user would simply need to select a button labelled ‘5’ and then select a second button labelled ‘DO.’

In still further embodiments, the system is configured to generate a report based at least in part on the observations and/or treatments entered into the charting system by the user. The system may, for example: (1) electronically retrieve, from a database, one or more pieces of text, descriptions, images, etc. for each particular observation and/or proposed treatment for each of the patient's teeth; and (2) generate a report that includes the one or more pieces of text, descriptions, images, etc. In particular embodiments, the report may include a visual representation of the patient's teeth with one or more markings indicating a location of particular observations and or proposed treatments. In particular embodiments, the report may include a plain language summary of observations made by the dentist comprising non-technical descriptions of proposed treatments (e.g., “We would recommend filling a cavity on your child's upper left canine tooth” as opposed to “Facial tooth 11”). In some embodiments, these plain language summaries may be predefined and stored in a database, associated with the one or more user entries to which they may pertain.

Exemplary embodiments of the Dental Charting and Data Management System 100 are discussed more fully below.

### Exemplary System Platform

Various embodiments of a Dental Charting and Data Management System 100 may be implemented in the context of any suitable system (e.g., a mobile computing system, dental practice computing system, etc.). For example, the Dental Charting and Data Management System 100 may be implemented to expedite the dental charting process by enabling a user to enter observations, recommended treatments, and other information related to a dental examination from a single, self-contained user interface.

Various aspects of the system's functionality may be executed by certain system modules, including a Dental Charting and Data Management Module 300. This module is discussed in greater detail below. Although this module is presented as a series of steps, it should be understood in light of this disclosure that various embodiments of the module described herein may perform the steps described below in an order other than in which they are presented. In still other embodiments, the module may omit certain steps described below. In various other embodiments, the module described herein may perform steps in addition to those described.

### Dental Charting and Data Management Module

In particular embodiments, when executing the Dental Charting and Data Management Module 300, the system begins, at Step 310, by creating an electronic record for an individual and digitally storing the record. In various embodiments, the system is configured to store the electronic record in computer memory (e.g., in a database or other suitable location). In some embodiments, the system may identify an existing electronic record for an individual (e.g., a patient), for whom a dental examination has been initiated.

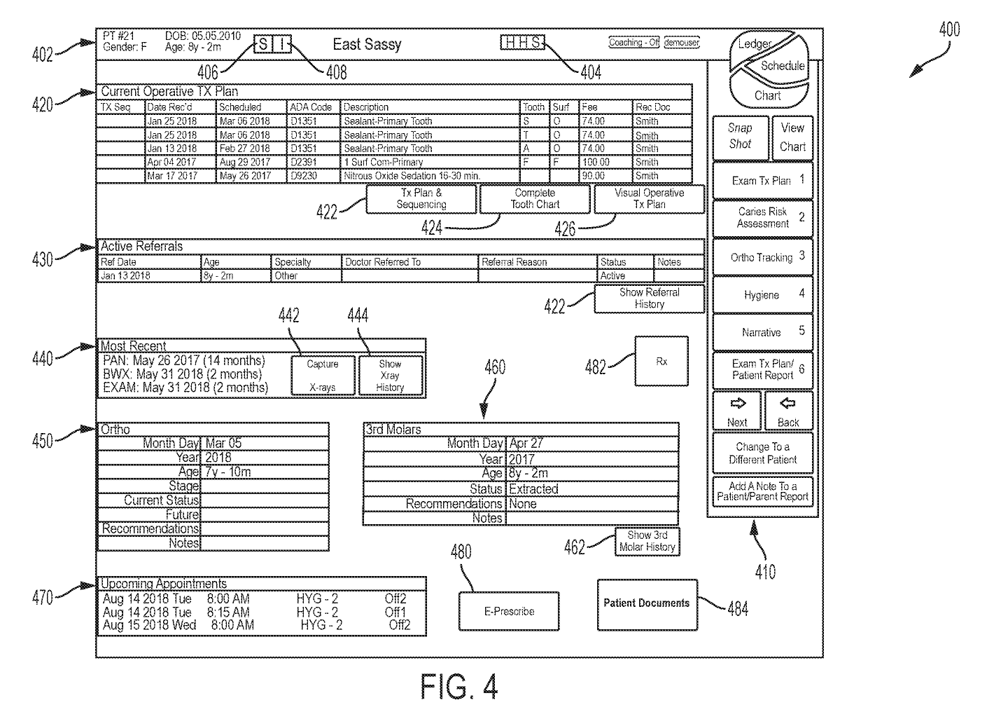

Continuing to Step 320, the system provides a graphical user interface for charting dental information for each of a plurality of teeth associated with the individual (e.g., patient). In some embodiments, the user interface comprises a user-selectable input (e.g., indicia, button, etc.) associated with each of the individual's plurality of teeth. In other embodiments, the user interface further comprises a plurality of user-selectable inputs (e.g., buttons, indicia, etc.) associated with various observations, proposed treatments, etc. that a dentist may make or propose during the course of an examination. In particular embodiments, each user-selectable input associated with the individual's teeth and the plurality of user-selectable inputs associated with the observations and treatments may be presented via the user interface on a single display screen. Examples of various graphical user interfaces will be shown and described in detail below with respect to FIGS. 4-17.

In various embodiments, each user-selectable input associated with the individual's teeth may comprise a number, letter, or other identifier that may enable the user to easily identify and select the input upon instruction by a dentist or other individual. In particular embodiments, a user that is aiding a dentist or dental hygienist in charting dental information using the system may not need to have any knowledge of tooth numbering conventions, dental terms, etc. in order to input information into the system using the interface.

In particular embodiments, the system continues to Step 330 where the system electronically receives input data comprising at least one observation and/or at least one proposed treatment for one or more of the individual's teeth (e.g., the patient's teeth). The system may, for example: (1) receive a selection, via the interface, of a first button associated with a first tooth (e.g., a first numbered tooth); and (2) receive selection, via the interface, of a particular button associated with a particular treatment or observation. The system may then continue to Step 340 where the system processes the input data and electronically associates the particular treatment or observation with the first tooth. The system may then repeat this process as the user continues to select different buttons representing different teeth, and subsequently selects one or more observations and/or treatments. The system may then electronically associate the input data for each tooth with the electronic record for the individual. The system may, for example, modify a data structure associated with the individual to include the newly submitted data for one or more of the individual's teeth. In various embodiments, each user-selectable button has one or more predefined data associated with the button (e.g., one or more of a preselected narrative, a preselected medical code, a preselected dental material, etc.). Therefore, when the user selects a tooth number and a treatment (e.g., tooth 12, DO) the system may also modify the data structure associated with the individual to include the preselected narrative associated with each selected button, the medical code associated with a button or combination of buttons, and/or the dental material. In this way, one or more reports (e.g., billing reports, insurance reimbursement reports, patient reports, treatment reports, etc.) may be generated based on the data structure associated with the individual.

In some embodiments, the system is configured to provide an error if the user attempts to enter, using the interface, an observation or treatment for a tooth that is not possible. For example, if a particular tooth does not have a mesial occlusal surface, the system may be configured to provide an error message if the user attempts to enter a treatment of placing a mesial occlusal filling on that particular tooth.

Returning to Step 350, the system may generate a summary report based at least in part on the input data. In some embodiments, the system may, for example: (1) retrieve one or more pieces of predefined text, images, etc. based at least in part on the input data; (2) compile the retrieved one or more pieces of predefined text, images, etc. into a summary report; and/or (3) provide the summary report (e.g., by displaying it, printing it, etc.) to any suitable individual (e.g., the patient, dentist, assistant, insurance company, etc.).

The system may, for example, store the one or more pieces of predefined text, images, etc. in a database and associate the one or more pieces of predefined text, images, etc. with particular inputs/pieces of data that the user may provide while using the system to chart dental information. For example, the system may store a sentence describing in layman's terms what is meant when a user indicates that tooth number 27 is erupting while charting the dental information using the system. If the user had provided that data input while charting data using the system (e.g., by selecting a button for tooth 27 and then selecting an observation indicating “erupting”), the system, when generating a summary report, would retrieve a text summary (e.g., predefined text) associated with such a selection for inclusion in the generated report.

### Exemplary User Experience

FIGS. 4 through 17 depict exemplary screen displays and graphical user interfaces (GUIs) according to various embodiments of the system, which may display information associated with the system or enable access to or interaction with the system by one or more users. As may be understood from these figures, a user may utilize the depicted user interfaces to, for example, input charting data into the system for a particular patient or review information about the particular patient. The user may navigate between the user interfaces via an interface menu 410. The various interfaces (which will be discussed more fully below) may include, for example, an interface for providing caries risk (e.g., cavity risks), an interface for orthodontic tracking, etc.

FIG. 4 shows a snap shot interface 400 according to various embodiments. The snap shot interface 400 may be the initial screen shown when a dentist, assistant, or other user retrieves a patient record in preparation for an exam or treatment. The snap shot interface 400 provides the dentist with a summary of information about the patient that is relevant to the patient's care. The summary includes information that is conventionally found in numerous locations in a patient's paper or electronic chart. The snap shot interface 400 provides the most relevant information from the patient's chart in one location that is easy to review prior or during a patient's examination. This information may include historical information regarding past visits, information regarding current or pending treatment, and information about future appointments. According to various embodiments, the information provided by the snap shot interface 400 is pre-populated when information is entered into the patient's record using any of the GUIs described herein, or using any other interface communicatively coupled to the Dental Charting and Data Management System 100 (e.g., an appointment and procedure scheduling system, a billing system, and patient management systems of other practitioners, such as orthodontists and oral surgeons performing related dental care to the same patient).

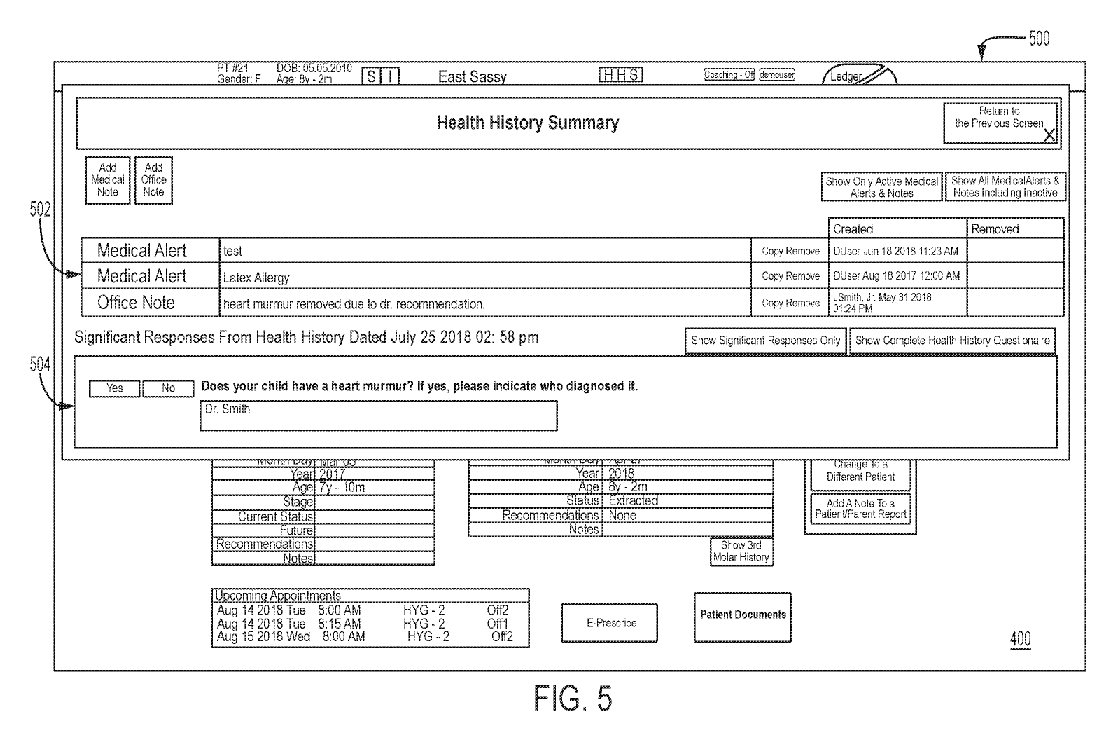

For example, according to the example shown in FIG. 4, the snap shot interface 400 includes patient demographics 402 along a header portion of the snap shot interface 400. The patient demographics 402 may include the name, a number, a gender, an age, and a birthdate associated with the patient. The header with the patient information 402 may be included on every screen of the Dental Charting and Data Management System 100 for quick reference by the user. According to one embodiment, the header also includes a health history summary (HHS) link 404 that, when selected, navigates the GUI to the HHS interface shown and described below with respect to FIG. 5.

The header may include additional links that may be commonly used to readily navigate to other patient information. For example, the header may include a schedule link 406 to navigate to a screen having appointment scheduling information corresponding to the patient from other screens, and an insurance link 408 to navigate to a screen having insurance information corresponding to the patient. It should be appreciated that any number and type of links may be included in the header or other portion of the GUIs described herein without departing from the scope of this disclosure. Similarly, the header may include any type of information that would be beneficial for display across all GUIs provided by the system.

As mentioned above, the snap shot interface 400 includes a summary of information about the patient that is relevant to the patient's care, which is conventionally found in numerous locations in a patient's paper or electronic chart. The snap shot interface 400 may be used by the dentist or other practitioner to familiarize himself or herself with the patient's history and current status prior to an examination or procedure since the snap shot interface 400 provides a summary of care and relevant patient information all in one screen.

According to the embodiment shown in FIG. 4, the snap shot interface 400 includes information sections corresponding to a current operative treatment plan section 420, an active referrals section 430, a most recent examination section 440, an orthodontics section 450, a third molars status section 460, and a future appointment section 470. It should be appreciated that the snap shot interface 400 is not limited to the various sections and links described herein with respect to FIG. 4. According to one embodiment, the snap shot interface 400, and all other interfaces shown and described with respect to FIGS. 5-17, are customizable according to the desires of the associated dentist or medical practitioner. The sections and links shown with respect to FIG. 4 provide a dentist using the system with a single location that provides the examination and treatment history, current treatment plan, referral and orthodontics information, and future scheduled appointments associated with a patient. Additional and more detailed information than that provided by the snap shot interface 400 may be located using any of the links described below, as well as with the menu 410.

The current operative treatment plan section 420 of the snap shot interface 400 includes information regarding treatments that have been recommended, but not yet completed. This information might include sequencing information if there are multiple treatments to be performed, dates that the plan was received and scheduled, American Dental Association (ADA) codes corresponding to the treatments, descriptions of the treatments, codes corresponding to the tooth and surface to be treated, corresponding fees, and the name of the recommending dentist. Any desired treatment plan information may be provided in the current operative treatment plan section 420 without departing from the scope of this disclosure. According to one embodiment, links 422, 424, and 426 may be provided corresponding to additional interface screens for inputting or reviewing information about treatment plan and sequencing, a complete tooth chart for the patient, and a visual operative treatment plan, respectively. These and other links may navigate the user to other screens providing graphical user interfaces shown and described below, or similar screens to those described herein.

The active referrals section 430 shows information regarding any referrals that have been provided to the patient to specialists or other practitioners. For example, if the dentist has referred the patient to an oral surgeon for removal of third molars, that information may be presented in this section. Additional referral information may be accessed via the referral history link 422, if any additional information exists for this patient. The most recent examination section 440 provides dates or other information about the most recent radiographs and examinations for this patient. For example, FIG. 4 shows that East Sassy last had panoramic dental radiographs 14 months ago, bitewing radiographs 2 months ago and the last exam 2 months ago, which was on May 31, 2018. The dentist may view those radiographs or further radiograph history by selecting links 442 and 444, respectively.

The orthodontics section 450 shows the orthodontics status of the patient and any related information. The third molars status section 460 shows the status of the patient's third molars, such as if and when they were extracted. Further information may be reviewed by selecting the third molar history link 462. The future appointment section 470 informs the dentist or user of any upcoming appointments that have already been scheduled.

The snap shot interface 400 also may provide additional information than described above, or links to additional screens that provide additional information or graphical user interfaces for receiving input. For example, the dentist may select the E-Prescribe link 480 to navigate to an interface that allows the dentist to electronically prescribe medication for an individual, directly linking to one or more pharmacies. Alternatively, the dentist may select the Rx link 482 to print out a prescription for a patient. Additional patient documents may be found by selecting link 484, which may link to a menu of available documents stored in the One or More Databases 140.

As discussed above, the header of the various graphical user interfaces of the Dental Charting and Data Management System 100 may include an HHS link 404 that, when selected, navigates the GUI to the HHS interface. An example HHS interface 500 is shown in FIG. 5. According to this example, the HHS interface 500 is a pop-up that is provided on top of the snap shot interface 400 or other screen on which the user is currently viewing. However, it should be appreciated that the HHS interface 500 may be a stand-alone screen that does not pop up over the screen on which the HHS link 404 is selected.

The HHS interface 500 provides the dentist with any allergy or other medical alert 502 that may be relevant to the treatment of the individual. In the example shown, the dentist may quickly click on any HHS link 404 and see that the patient has a latex allergy and that the patient had a diagnosed heart murmur in the past. According to various embodiments, patients are provided with an electronic health history questionnaire that allows the patients to input all of their medical and family history. Certain questions that are deemed medically relevant that receive a “yes” response or additional information input from the user may be deemed a significant response 504 and is shown on the HHS interface 500. The information regarding the heart murmur in FIG. 5 is an example of a significant response 504 that is readily available to the dentist. The dentist may select various inputs to show only the significant responses 504, shown the complete questionnaire, add medical and office notes, and to filter the information shown on the HHS interface 500.

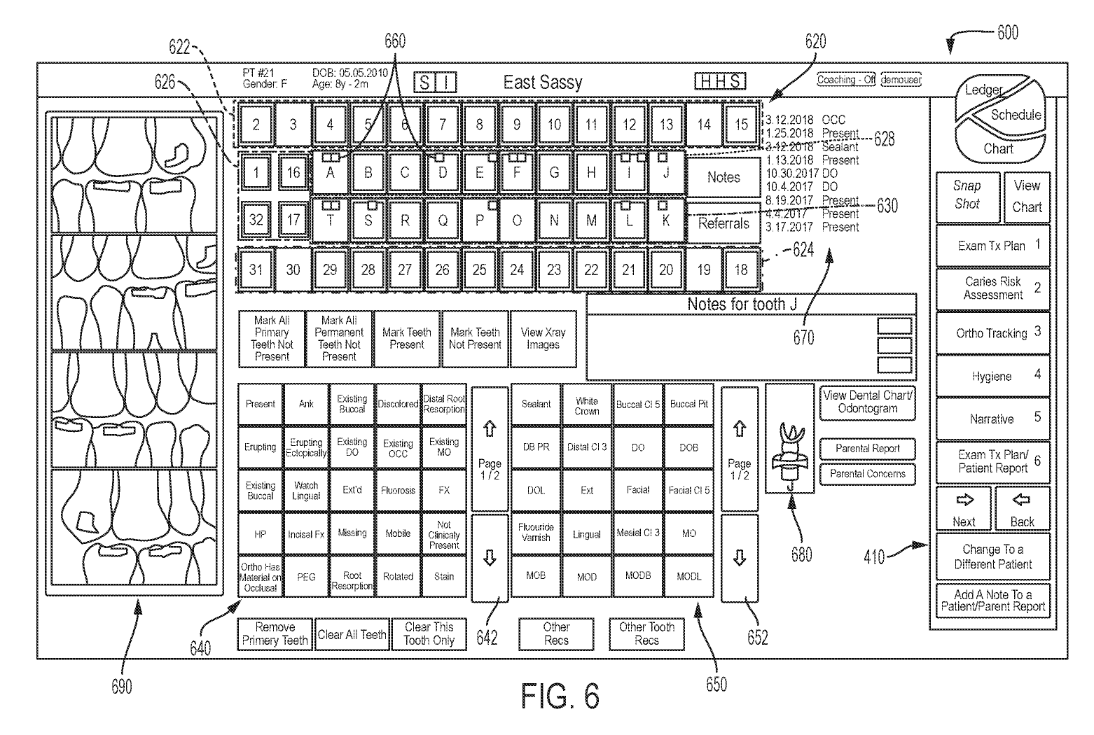

FIG. 6 depicts a dental charting interface 600 according to a particular embodiment. As may be understood from FIG. 6, the dental charting interface 600 includes user-selectable inputs (e.g., buttons or indicia) for each of a particular patient's teeth 620. These inputs include, for example: (1) upper permanent teeth inputs 622; (2) upper primary teeth inputs 628; (3) lower permanent teeth inputs 624; (4) third molars inputs 626; and (5) lower primary teeth inputs 630. As shown in this figure, the upper permanent teeth inputs 620, lower permanent teeth inputs 624, and third molars inputs 626 comprise numbered, user-selectable buttons. In particular embodiments, the numbers of each input may correspond to a particular tooth. In some embodiments, the numbers of each tooth substantially correspond to (e.g., correspond to) the universal numbering system used for numbering teeth (e.g., the tooth designated “1” is the maxillary right third molar (“wisdom tooth”) and the count continues along the upper teeth to the left side. Then the count begins at the mandibular left third molar, designated number 17, and continues along the bottom teeth to the right side.). In various embodiments, the permanent teeth may be referred to as adult teeth.

The primary teeth (e.g., baby teeth) are similarly labelled, using capital letters A through T. By including inputs for both permanent and primary teeth, the user interface may, for example, enable a user to input charting information for a patient that still has a mix of primary and permanent teeth (e.g., has not lost all of their primary teeth). This configuration also allows the dentist to view all teeth (i.e., primary and permanent) simultaneously to view any observations that were made for the primary teeth even if the permanent tooth is now present without having to switch between views.

According to the embodiment shown in FIG. 6, each respective input for the individual's upper permanent teeth (upper permanent teeth inputs 622) are numbered from two through fifteen and displayed on the graphical user interface in numerical order from left to right in a first single row. Each respective input for the individual's upper primary teeth (upper primary teeth inputs 628) are lettered from ‘A’ through 1′ and displayed on the graphical user interface in alphabetical order from left to right in a second single row. Each respective input for the individual's lower primary teeth (lower primary teeth inputs 630) are lettered from ‘K’ through ‘T’ and displayed on the graphical user interface in reverse alphabetical order from right to left in a third single row. Each respective input for the individual's lower permanent teeth (lower permanent teeth inputs 624) are numbered from eighteen through thirty-one and displayed on the graphical user interface in reverse numerical order from right to left in a fourth single row.

The dental charting interface 600 further includes a plurality of observation inputs 640, which include a plurality of user-selectable buttons that correspond to different observations. As may be understood from FIG. 4, the plurality of observations from which a user may select may include, for example: (1) present; (2) erupting; (3) existing DO Amal; and/or (4) any other suitable observation related to a particular tooth. In particular embodiments, such as the embodiment shown in FIG. 6, the dental charting interface 600 further comprises one or more scroll buttons 642 that a user can select to scroll through additional observations to select. In various embodiments, the displayed observations are customizable (e.g., a particular user may submit particular observations for inclusion in the interface and later selection).

The dental charting interface 600, in the embodiment shown in FIG. 6, further comprises a plurality of treatment inputs 650, which include a plurality of user-selectable buttons that correspond to different treatments. As may be understood from FIG. 6, the plurality of treatments from which a user may select may include, for example: (1) Check in 3 Months; (2) Facial (e.g., to indicate placing a filling at that particular location on a tooth); (3) Fluoride Varnish; and/or (4) any other suitable treatment related to a particular tooth. In particular embodiments, such as the embodiment shown in FIG. 6, the dental charting interface 600 further comprises one or more scroll buttons 652 that a user can select to scroll through additional treatments to select. In various embodiments, the displayed treatments are customizable (e.g., a particular user may submit particular treatments for inclusion in the interface and later selection).

As may be understood from FIG. 6, when entering charting data for a particular patient, the user may: (1) select a first tooth (e.g., tooth ‘5’ from the permanent teeth inputs 622): (2) select an observation from the observation inputs 640 (e.g., present); and/or (3) select a treatment from the treatment inputs 650 (e.g., Facial, to indicate that a filling is required on the facial portion of tooth ‘5’). The user may make these selections using any suitable input device (e.g., a mouse or keyboard), using their finger via a touch-screen display on which the dental charting interface 600 is displayed, etc.

The dental charting interface 600 may additionally include a visual representation of at least one dental radiograph 690 corresponding to the plurality of teeth 620 associated with the individual. Conventionally, a dentist would have to navigate to a separate screen or system to view dental radiographs. By embedding the most current radiographs into the dental charting interface 600, the dentist conveniently can examine the patient's actual teeth while having quick access to a view of the radiographs 690 on the dental charting interface 600, which is used to enter observations and treatments recommendations.

In some embodiments, the user is able to use the dental charting interface 600 to input information stated by a dentist during a dental exam. The dentist may, for example, go through each of the patient's teeth, announcing a number or letter corresponding to each tooth followed by one or more observations or treatments. As the dentist progresses through the teeth, the system enables the user (e.g., an assistant) to quickly and easily input the information into the system using the dental charting interface. The system may, for example, associate any observation or treatment selected by the user (e.g., using the treatment inputs 650 or observation inputs 640) with the most recently selected tooth (e.g., any of the upper permanent teeth, upper primary teeth, lower permanent teeth, third molars inputs, or lower primary teeth). As may be understood from this figure, the simple, straightforward nature of the interface may enable an untrained assistant with no prior dental knowledge to input information by simply selecting numbered or lettered buttons at the direction of the dental professional (e.g., dentist, dental hygienist, etc.).

Another feature of the dental charting interface 600 is a graphical representation of the particular tooth 680 that is associated with the input 620 for the specific tooth being observed and/or treated. After the dentist or assistant has selected the appropriate input 620 corresponding to the desired tooth, the visual representation of the particular tooth 680 is shown on the dental charting interface 600, along with a representation of the observation associated with the selected observation input 640 and/or the treatment corresponding to the treatment input 650. In the example shown in FIG. 6, the particular tooth 680 is shown with a green band representing a specific issue (like tarter) to watch on a particular face of that tooth. Similarly, according to one embodiment, each tooth input 620 may include an information indicator 660 that gives the dentist a visual indication that the particular tooth has some type of information associated with it. For example, a small green box as the information indicator 660 may represent that the tooth has some issue that the dentist would like to watch or make note of A small red box as the information indicator 660 may represent that the tooth has a treatment that has not yet been completed. A small blue box as the information indicator 660 may represent that the tooth has a treatment that has previously been completed.

These information indicators 660 may be input using the box titled “Notes for tooth J” in FIG. 6, or when the user selects an observation input 640 or a treatment input 650. This box provides a location to input information about a particular tooth selected by a tooth input 620 (in the example shown, tooth J has been selected). Textual information 670 may provide textual information (e.g., date, observation, treatment) regarding a selected tooth. In this example, the dates and corresponding observations and treatments are shown for the selected tooth J. This textual information may be color coded to represent observation or treatment, similar to the information indicators 660 and the representations depicted with respect to the visualization of the particular tooth 680.

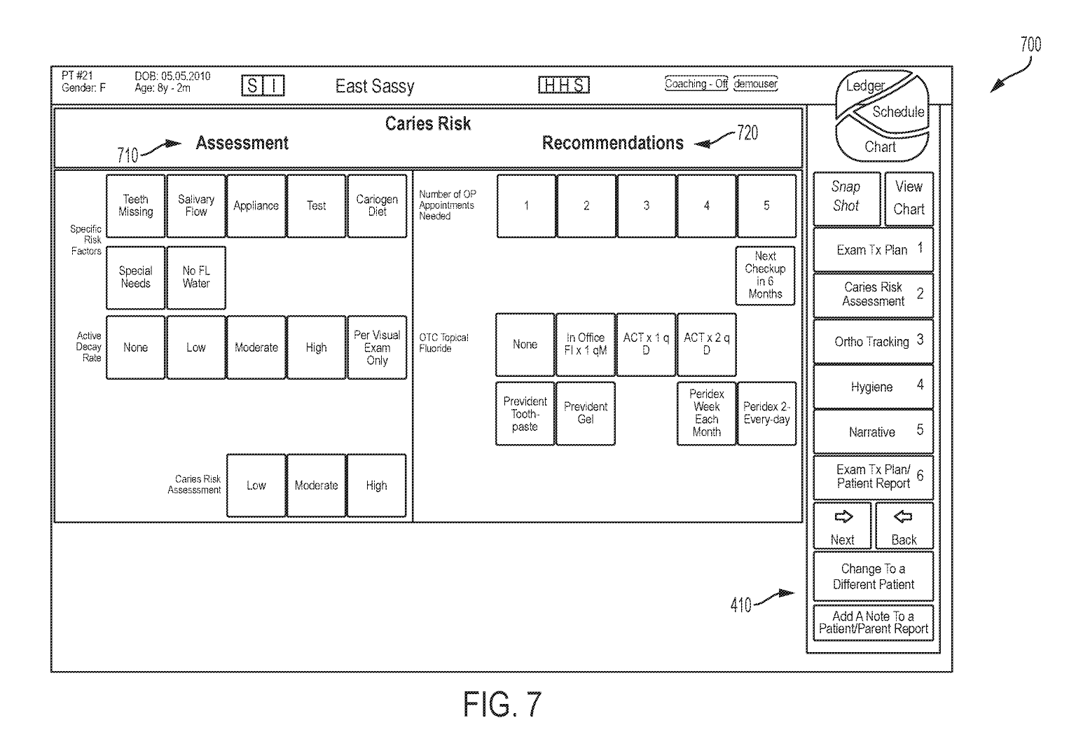

Continuing to FIG. 7, the system further includes a caries risk interface 700 with which the user may enter information related to one or more assessments and/or recommendations to one or more caries identified (e.g., or not identified) during the dental exam of the patient (e.g., by a dentist). As may be understood from this figure, the caries risk interface 700 comprises a plurality of user-selectable assessment inputs 710, which may include for example: (1) one or more specific risk factors related to carries (e.g., diet, special needs, non-fluoridated water, etc.); (2) an active decay rate (e.g., none, low, moderate, high, etc.); (3) an overall caries risk assessment (e.g., low, moderate, high, etc.); and/or (4) any other suitable inputs with which a user may provide information related to caries risk assessment. In addition to the assessment inputs, the caries risk interface 700 comprise one or more user-selectable recommendation inputs 720, which may include (1) the number of operatory appointments need; (2) whether the patient should use any topical fluoride, and/or (3) other suitable inputs for other recommendations (e.g., need for flossing, next checkup, etc.).

As may be understood from this figure, the pre-selectable buttons related to these risk factors and assessment observations may facilitate the quick, easy, and straight-forward entry of an overall assessment for the patient by the user (e.g., based on the dentist's observations). It should also be understood that each of the listed buttons may also be associated with one or more predefined text entries that are entered into the patient's database record structure as the data is entered by the user. In this way, reports and other narratives may be generated by extracting certain data from the patient's data record.

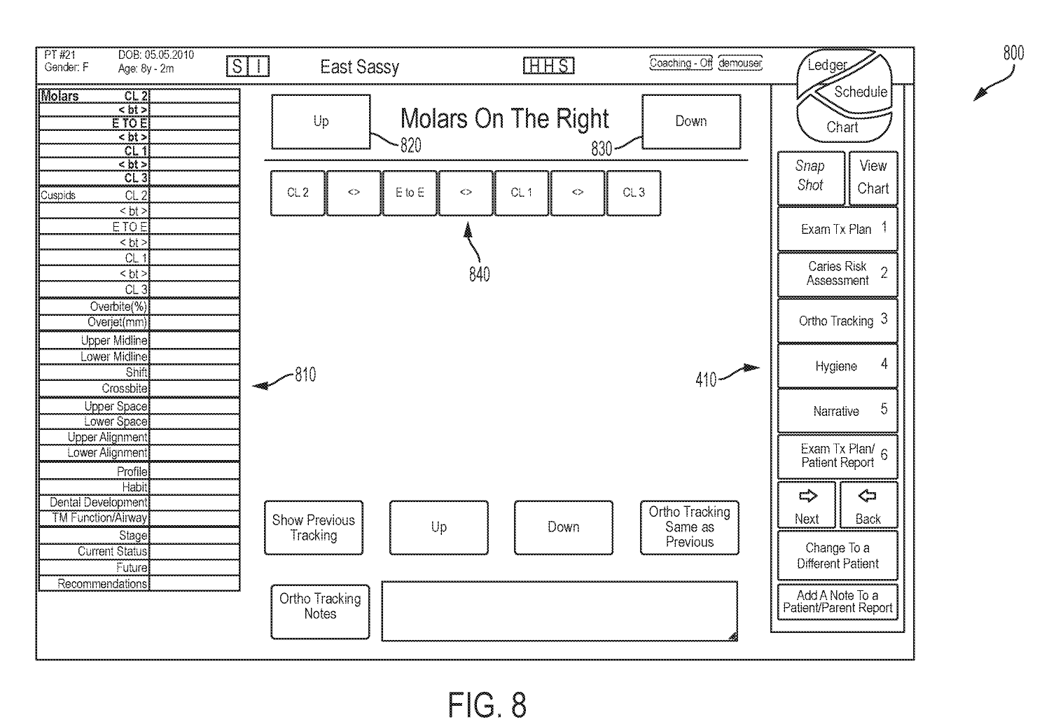

FIG. 8 depicts an ortho-tracking interface 800. As may be understood from this figure, the user may enter measurements and observations related to orthodontics (e.g., tooth measurements, overbite, overjet, upper and lower midline measurements, crossbite, tooth angles, etc.). In various embodiments, a listing of the various measurements may be displayed in a table 810. The user can navigate through the various rows in the table using an up or down input button 820 and 830. As the user moves up or down through the displayed measurement table 810, different user selectable buttons 840 are displayed to the user in order to allow the user to input measurements provided by the dental professional. For example, in FIG. 8, the interface is configured to enter information about the patient's molars. The system may receive and store any provided measurements and observations in a database associated with a particular patient. The system may be further configured to track measurements and observations made over a series of dental examinations, for example, to track progress of the patient's teeth following an extraction, while the patient has braces, etc. For example, the dental professional can track how fast the patient's upper and lower midline is moving, etc.

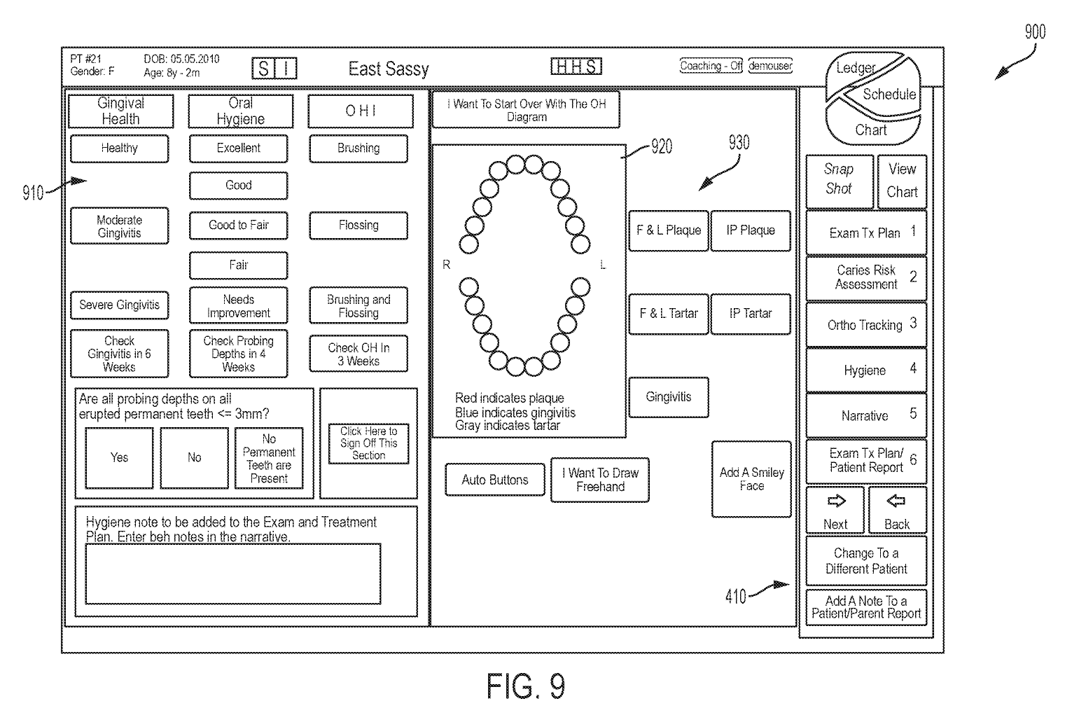

FIG. 9 depicts an exemplary hygiene interface 900 according to a particular embodiment. As may be understood from this figure, the hygiene interface 900 comprises a plurality of user-selectable buttons related to oral hygiene 910. For example, the hygiene interface comprises a plurality of selectable buttons related to gingival health, oral hygiene, and oral hygiene instructions (e.g., OHI). During the dental examination of a patient, the user can make selections via the hygiene interface 900 related to the patient's dental hygiene. The user may, for example, select that the gingival health of the patient is ‘healthy,’ the oral hygiene of the patient is ‘good,’ and that the patient should focus on ‘brushing’ as shown. In various embodiments, the user inputting the observations may be an assistant to a dentist, a dental hygienist or the dentist themselves.

The hygiene interface 900 in FIG. 9 further includes an oral diagram 920 on which the user can make indications of locations of plaque, tartar, and gingivitis. As may be understood from this figure, the user may select a button 930 for the type plaque, tartar, or gingivitis that the user would like to indicate on the oral diagram 920. The user may then draw on the oral diagram to indicate the location of the selected substance or observation. Each type of plaque, tartar or gingivitis may be associated with a different color, so that there is a clear distinction between what has been marked when the system generates a report as discussed below. The system is also configured to allow the user to draw freehand using a touch screen or mouse. As with other user-selectable input buttons, each button may have predefined text associated with the button that is stored in the patient's database record. The predefined text may be used by the system to create the dental history narrative as well as in generating a patient treatment record. Thus, each user-selectable button may have a first predefined text narrative that is drafted in medical terminology and a second predefined text narrative that is drafted in layman terms. In some embodiments, the first predefined text narrative may be used in the medical records and reimbursement request reports and the second predefined text narrative may be used in generating the patient's treatment report.

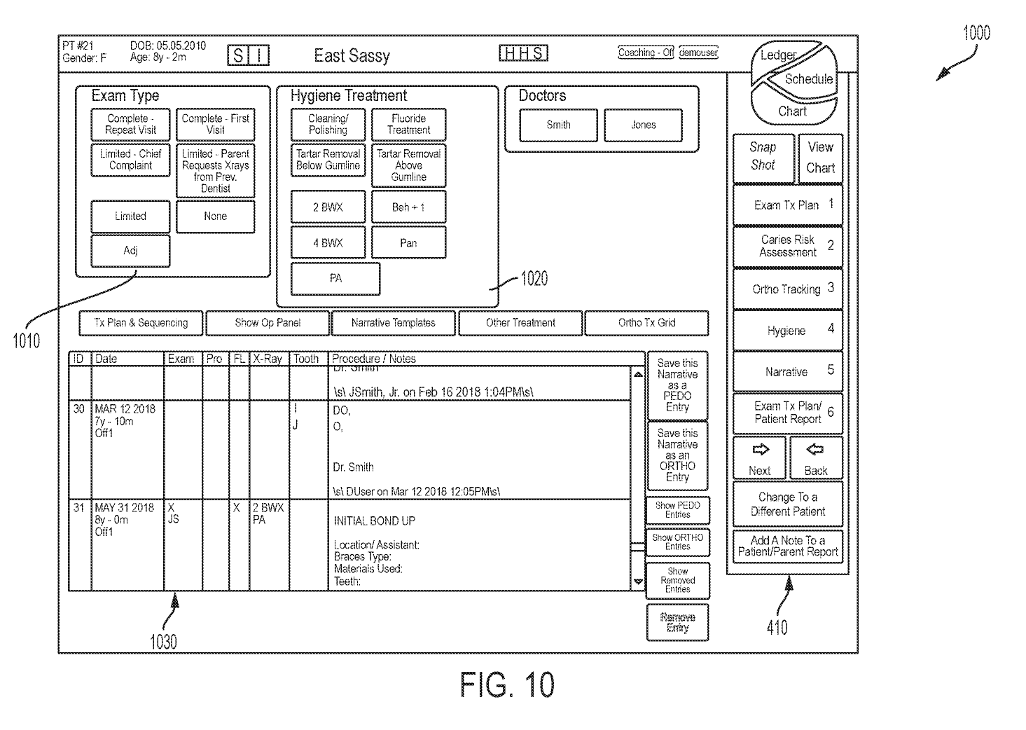

Continuing to FIG. 10, the system, in various embodiments, further comprises a narrative interface 1000 via which a user can submit information related to what sort of exam was performed on the patient, what sort of hygiene treatment was administered, restorative treatment completed, etc. As shown in this figure, the narrative interface 1000 comprises a plurality of user-selectable exam type buttons 1010 from which a user may select what type of exam was performed on the patient. This may include, for example: (1) Complete Exam—Repeat Visit; (2) Complete Exam—First Visit; (3) Limited Exam—Chief Complaint; and/or (4) any other suitable type of exam that may have been administered (e.g., orthodontic consultation, radiographs, follow-up, etc.).

The narrative interface 1000 further comprises a plurality of user-selectable hygiene treatment buttons 1020, from which a user may select one or more types of treatment administered during the patient's visit. These may include, for example: (1) cleaning/polishing; (2) tartar removal; (3) bitewings (e.g., radiographs); and/or (4) any other suitable treatment. As shown in this figure, the narrative interface 1000 further depicts past visit data 1030. The system may, when displaying the narrative interface 1000: (1) retrieve past visit narrative/treatment data for the patient; and (2) display the data via the narrative interface 1000.

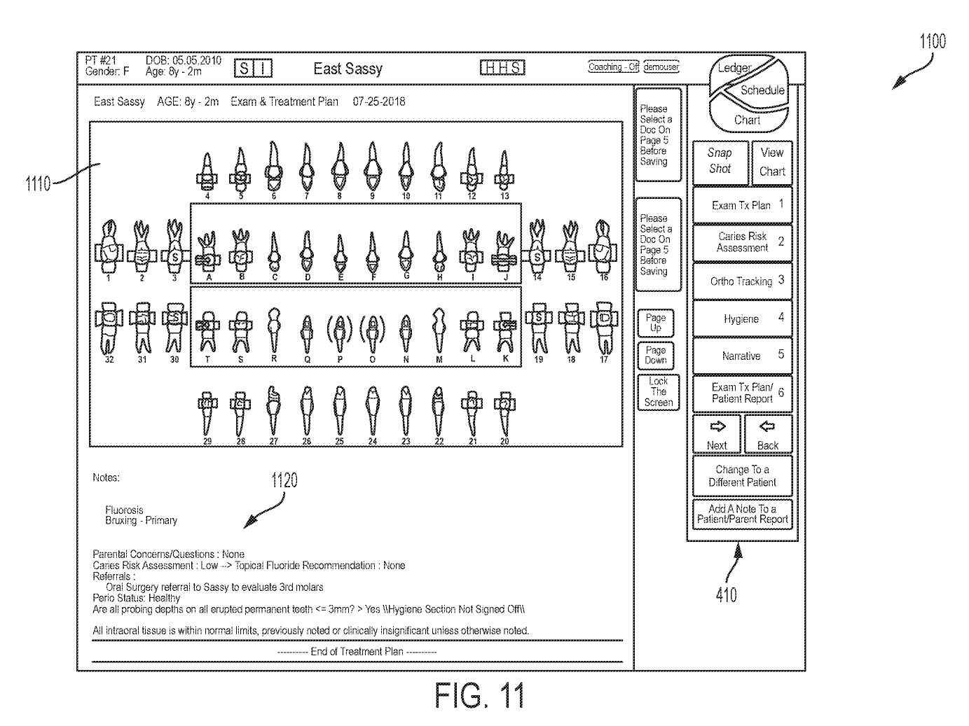

FIG. 11 depicts an exam and treatment plan interface 1100 according to a particular embodiment that comprises information received by the system via the various user interfaces discussed above. As shown in this figure, the system may generate the exam and treatment plan interface 1100 based on one or more pieces of data provided by the user. For example, the exam and treatment plan interface 1100 includes a visual representation of the patient's teeth 1110, which includes indications in the form of markings that show potential problem areas or areas indicated as requiring a filling. These markings may be color coded to represent observations, past or recommended treatments, or any other desired information that would benefit from color coding.

In the example shown, each tooth may indicate the presence of caries, fillings, crowns, bridges or other dental work or disease associated with the individual tooth. For example, in the visual representation shown in this figure, teeth A and K are marked with fillings. The location of these fillings may substantially correspond to a location entered by the user for those teeth using the charting interface 600 shown in FIG. 6. For example, the system may generate the visual representation of the user's teeth 1110 such that it includes markings in locations indicated during charting. For example, if a particular tooth selected during charting was indicated as requiring a mesial occlusal filling (e.g., because the user selected tooth A and then ‘MO’ via the charting interface 600), the system would generate a visual representation of the teeth 1110 that includes a marker on the mesial occlusal portion of the tooth.

As may be understood from this figure, the system may generate a visual representation of a patient's teeth showing a proper location of any observations based simply on selection, by a user, of a number (e.g., representing the tooth) and an acronym or word (e.g., MO). As discussed above, this may enable the system to produce such a visual report without requiring the user to have any knowledge of teeth locations, teeth surfaces, etc. In some embodiments, the system may store a single image of each tooth with each particular surface and location marked. In other embodiments, the system may store a plain picture of each tooth and superimpose location markings over the image.

The exam and treatment plan interface 1100 further includes notes 1120, which may include, for example, one or more free-form observations entered by the user (e.g., “most of Davis' teeth show signs of being chipped”), and other information entered using the various interfaces discussed above (e.g., a caries risk assessment). In various embodiments, the notes section may be created based on the predefined text narrative (e.g., the first predefined text narrative and/or the second predefined text narrative) associated with the user-selectable buttons that were selected by the user during the examination or treatment. It should be noted that the information shown in the exam and treatment plan interface 1100 is pre-populated from input provided during the examination using the dental charting interface 600 and other interfaces, without requiring a user to manually enter the information on this chart as is done in conventional dental charting systems.

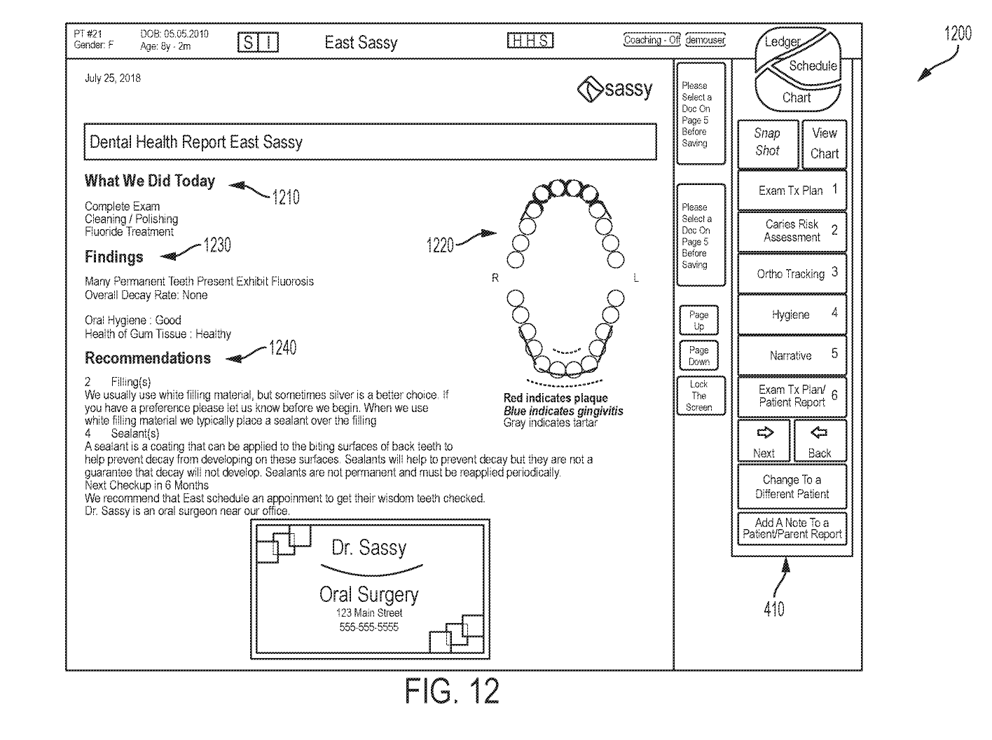

FIG. 12 depicts a patient report interface 1200 according to yet another embodiment. As may be understood from this figure, the patient report interface 1200 in the example shown may include a plain language summary (i.e., in layman terms) of various observations and treatments made during a particular visit in addition to recommendations, etc. The patient report interface 1200 shown in FIG. 12 includes treatment description 1210 (e.g., “What We Did Today”), a diagram of the patient's teeth 1220 that indicates plaque, tartar and gingivitis, observations 1230 (e.g., “Findings”), and recommended treatments 1240.

In various embodiments, the system may generate the patient report shown in the patient report interface 1200 that is based at least in part on one or more inputs provided via the user interfaces discussed above. In particular embodiments, particular interface entries are associated with one or more predefined descriptive text (e.g., the first predefined text narrative in medical terms and/or the second predefined text narrative in layman terms) that conveys an easy to understand summary of various observations made, treatments performed, and recommendations based on a dental examination. Thus, most if not all, of the text in the patient report comes from information stored in the patient's database record, which are generated as the user makes selections from the exam interface 600, the caries risk interface 700, the ortho-tracking interface 800, the hygiene interface 900, and the narrative interface 1000. In various embodiments, such a patient report 1200 may be suitable for presentation to a patient or parent of a young patient (e.g., because the patient report 1200 presents data in non-technical terms which a patient may be better able to understand).

For example, in FIG. 9, the hygiene interface 900 shows a plurality of user-selectable buttons related to oral hygiene 910 with “Brushing” selected as an oral hygiene instruction. This may indicate, for example, that the patient should be instructed to focus on or otherwise improve their brushing. The system, after storing this selection in association with the patient, may generate a patient report 1200 such as the patient report shown in FIG. 12. When generating the patient report 1200, the system may retrieve descriptive text for all entries made using the user interfaces discussed above, and combine those descriptive text entries into a generated report. In this example, the system may retrieve the phrase “We went over brushing techniques today” for inclusion in the patient report 1200 based on the selection of “Brushing” as discussed above with respect to FIG. 9 (e.g., because that phrase is stored in memory such as a database and associated with the selection of “Brushing” in the system).

As may be understood from FIG. 12, the patient report 1200 may include a plurality of fields from which the system may retrieve pre-coded text or image entries based on data submitted during a dental examination (e.g., via the user interfaces described above). The system may, for example, automatically populate a template patient report (e.g., which includes some generic description and headings) with specific information about a particular patient, a particular examination, etc. The system may, for example, retrieve and populate the treatment description 1210 with text associated with particular data entries made by the user. Similarly, the system may retrieve the diagram of the patient's teeth 1220 that indicates plaque, tartar and gingivitis markings made using the hygiene interface 900 shown in FIG. 9.

In particular embodiments, the generated report may include information regarding one or more referrals made during the examination (e.g., see Dr. Johns for endodontic care; see Dr. Roof, oral surgeon, for third molars removal, etc.) so that the patient has a written record of the referral. This is especially important when the patient is a minor or one parent needs to convey the information to another parent. The referral information may include, for example, contact information, the reason for the referral, and any other information necessary to communicate to the referred doctor. As an example, FIG. 12 shows a box at the bottom of the patient report 1200 that provides information for Dr. Sassy, an oral surgeon that the patient is being referred to. In other embodiments, the report may include information regarding costs of proposed treatments. As above, this information may be populated from predefined text associated with the particular proposed treatments.

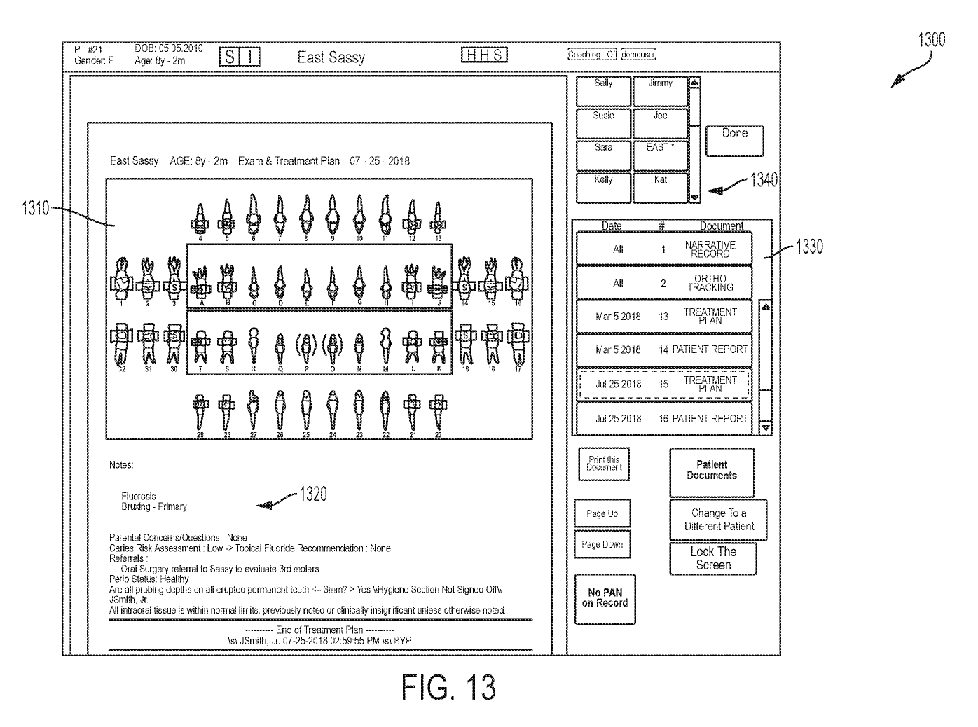

FIG. 13 shows a family history interface 1300 that allows the dentist to view current and historical information about not only the patient being seen, but also other members of the patient's family. According to one embodiment, selecting the “View Chart” link from the menu 410 in any of the screens navigates the user to the family history interface 1300. FIG. 13 shows an example of the Exam and Treatment Plan for patient East Sassy. The Exam and Treatment Plan shows a visual representation of the patient's teeth 1310 (similar to the visual representation 1110 discussed above with respect to FIG. 11) and the corresponding notes 1320 (similar to notes 1120 above). However, on the right side of the family history interface 1300, there are family inputs 1340 corresponding to all siblings or other family members associated with this patient, East Sassy. As seen in this example, East Sassy has family members, Sally, Jimmy, Susie, Joe, Sara, Kelly, and Kat. The dentist may be able to quickly locate all information pertaining to all family members by simply selecting the appropriate family input 1340. This feature is useful when the dentist wants to investigate whether issues are common to all family members, whether one or more family members have upcoming appointments in order to coordinate a required appointment for the existing patient, or when a parent requests information about multiple children. The family history interface 1300 additionally includes a historical information section 1330 that includes links to historical documents or information about past appointments, treatment plans, reports, or any other applicable or desired documents.

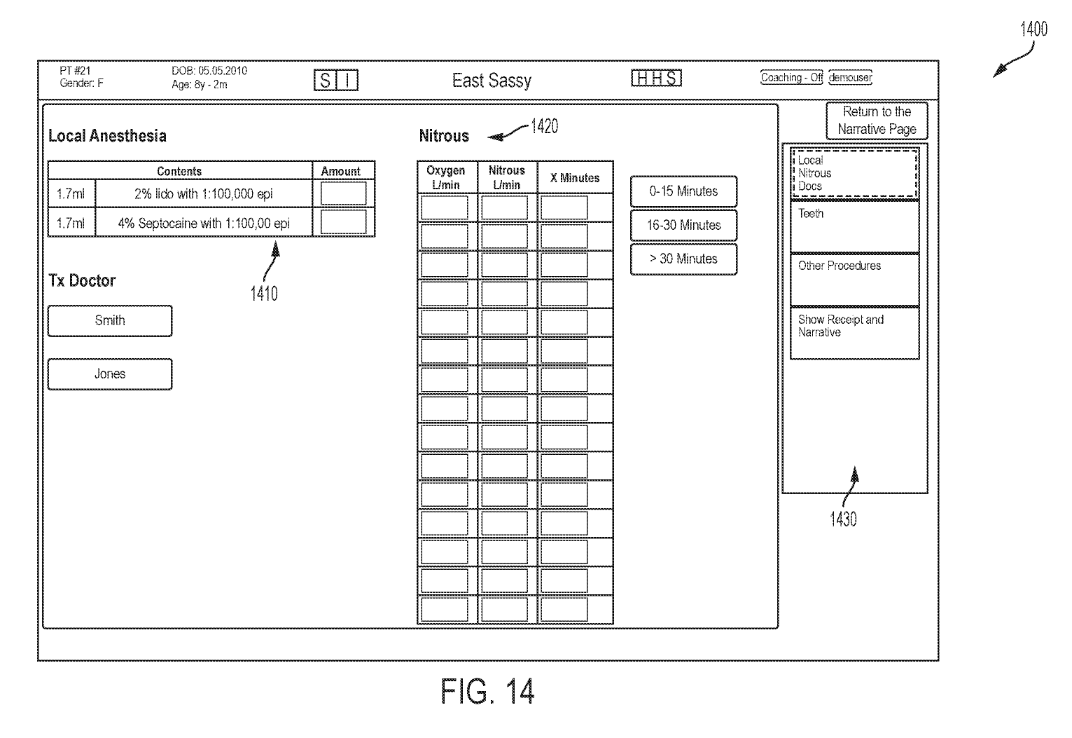

FIG. 14 shows an operative treatment interface 1400 in a series of post-treatment interfaces that are used by the dentist after the treatment of a patient to input further information and notes with respect to the treatment that was performed. The operative treatment interface 1400 shown in FIG. 14 allows the dentist to input information corresponding to the use of local anesthesia 1410 or nitrous oxide 1420. This information may be input manually or according to one embodiment, may be operatively connected to the nitrous oxide valve or mechanism to time and quantify the amount of nitrous oxide that is administered to the patient. A post-treatment menu 1430 allows the dentist or user to navigate between post-treatment interfaces.

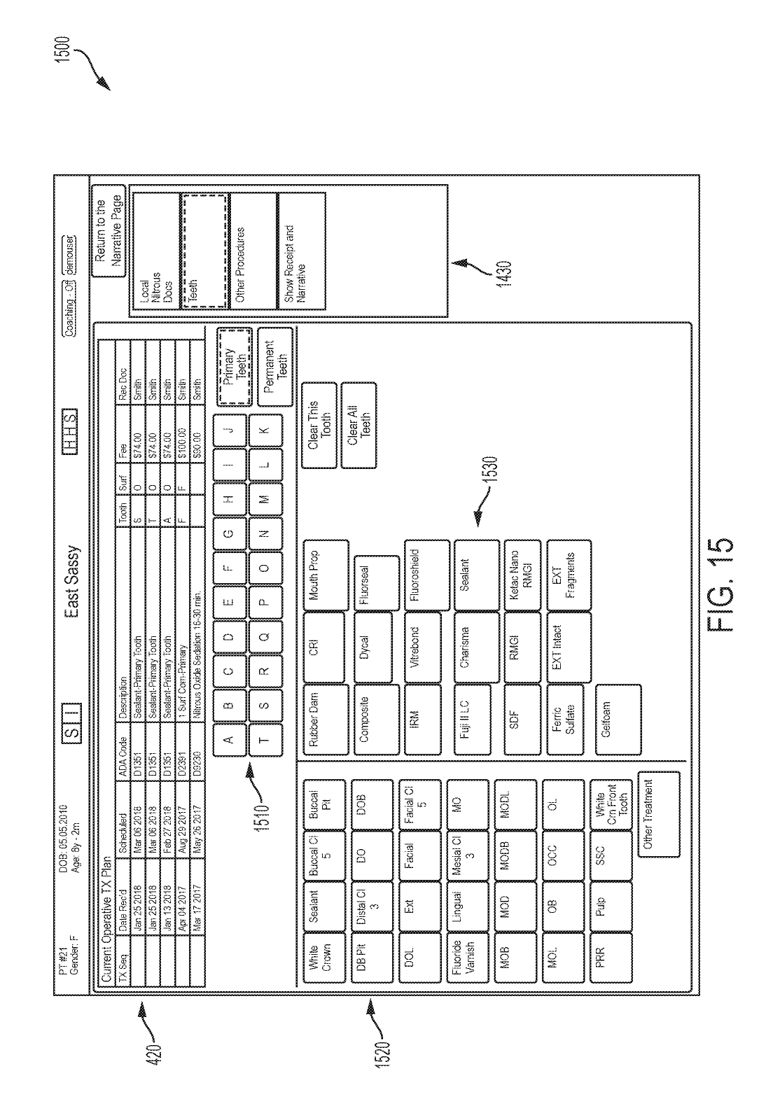

A post-examination treatment interface 1500 shown in FIG. 15 provides a graphical user interface for providing or editing information about treatments completed. The current operative treatment plan section 420 described above with respect to FIG. 4 is repeated on this interface for quick reference to the treatments that have been recommended, but not yet completed. The dentist or user may select the teeth, primary or permanent, with the tooth inputs 1510. There are a number of treatment inputs 1520, which are similar to the plurality of treatment inputs 650 described above with respect to the dental charting interface 600. The post-examination treatment interface 1500 additional includes a plurality of material inputs 1530 corresponding to the various materials that may have been used in the treatments selected using the treatment inputs 1520.

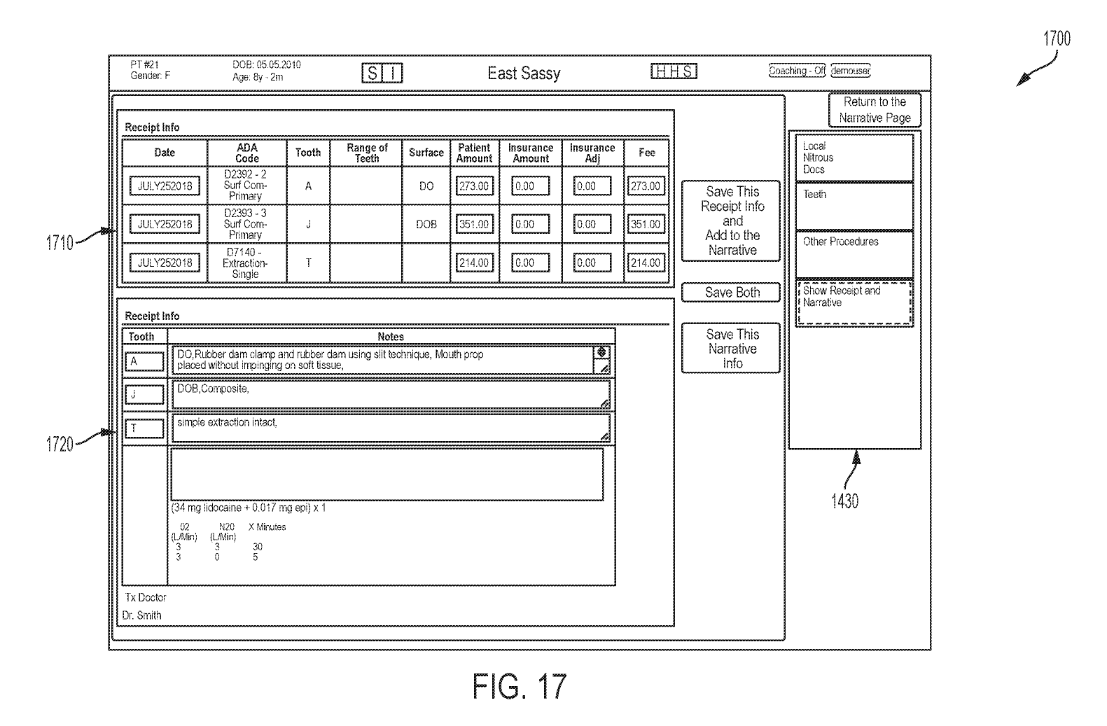

A user may also clear completed treatments from the current operative treatment plan section 420 using the post-treatment interface 1500. Selection of the various treatment inputs in the post-treatment interface 1500 associates treatments and materials with text and fees stored in the One or More Databases 140 and pre-populates the current operative treatment plan section 420 and the receipt interface discussed below with respect to FIG. 17.

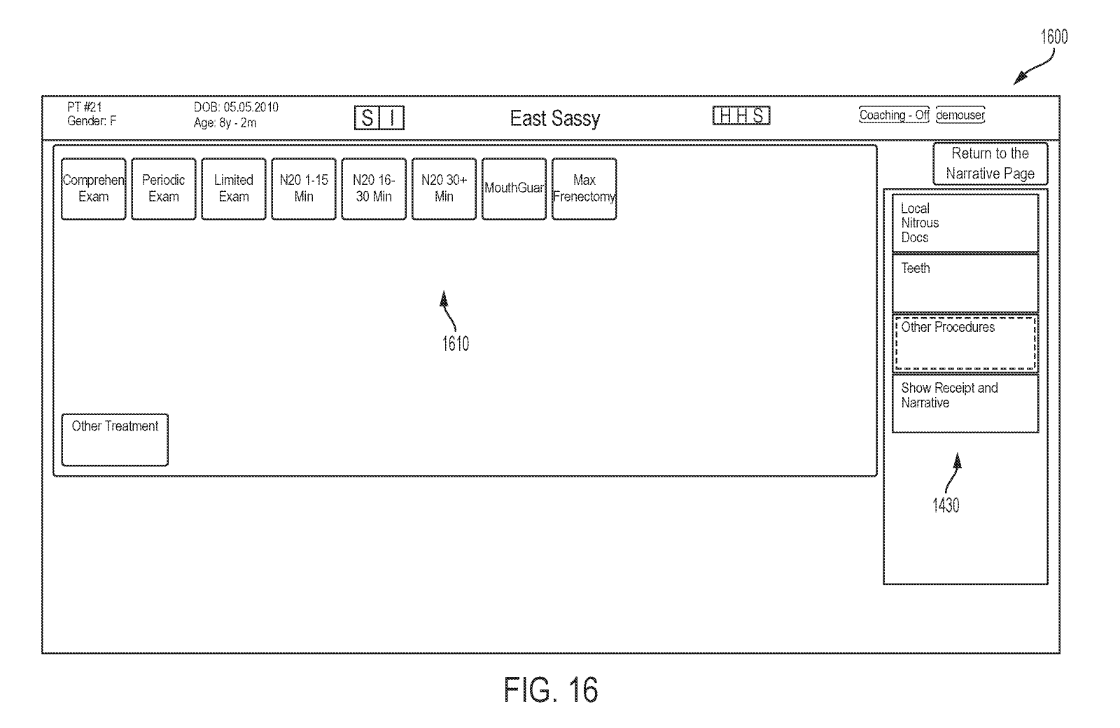

FIG. 16 shows a procedures interface 1600 used by the dentist or other user for providing information about procedures that were performed during the patient appointment. This interface may be customized with procedure inputs 1610 for common procedures that, when selected, pre-populate the electronic record of the patient, the patient report, and the billing information with any desirable narrative and cost data associated with the treatment.

FIG. 17 shows a receipt and narrative interface 1700. The receipt and narrative interface 1700 is pre-populated with information input in the post-treatment interface 1500 and any other applicable interface during or after the patient appointment. The receipt interface 1700 includes treatment and cost information 1710, as well as narrative information 1720 that provides notes about the various treatments. The narrative information 1720 may be pre-populated based on the treatment and materials that were selected previously and can be customized according to user preference. The receipt interface 1700 allows the dentist to review, edit, and save the receipt and narrative information to the patient's record, adding to the narrative notes shown in the past visit data 1030 of narrative interface 1000 and completing the examination and electronic charting procedure.

### CONCLUSION

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for the purposes of limitation.

What is claimed is:

1. A computer-implemented data processing method for electronically charting dental information for an individual, the method comprising:
creating an electronic record for the individual and digitally storing the record;
providing a graphical user interface for charting dental information for each of a plurality of teeth associated with the individual, the graphical user interface comprising:
a plurality of inputs corresponding to the plurality of teeth associated with the individual;
a plurality of observation inputs, each of the plurality of observation inputs being associated with a particular observation; and
a plurality of treatment inputs, each of the plurality of treatment inputs being associated with a particular treatment;
electronically receiving, input data comprising:
at least one first input of the plurality of inputs, the at least one first input comprising a selection of a particular tooth of the plurality of teeth; and
at least one second input associated with the at least one first input, the at least one second input comprising an input selected from a group consisting of:
at least one observation; and
at least one treatment;
processing the input data by:
electronically associating the particular tooth with the at least one observation or the at least one treatment; and
electronically associating the input data with the electronic record for the individual;
generating, based on the input data, a summary report of the at least one observation or the at least one treatment for the particular tooth, the summary report comprising:
a plain-language description of the at least one observation or the at least one treatment; and
a diagrammatic representation of the particular tooth relative to the plurality of teeth.

2. The computer-implemented data processing method of claim 1, wherein the graphical user interface is configured to display the plurality of inputs corresponding to the plurality of teeth associated with the individual, the plurality of observation inputs, and the plurality of treatment inputs on a single display screen.

3. The computer-implemented data processing method of claim 1, wherein the plurality of inputs for each of the plurality of teeth associated with the individual comprises:
a respective input for each of the individual's upper primary teeth;
a respective input for each of the individual's upper permanent teeth;
a respective input for each of the individual's lower primary teeth;
a respective input for each of the individual's lower permanent teeth; and
a respective input for each of the individual's third molars.

4. The computer-implemented data processing method of claim 3, wherein:
each respective input for the individual's upper permanent teeth are numbered from two through fifteen and displayed on the graphical user interface in numerical order from left to right in a first single row;
each respective input for the individual's upper primary teeth are lettered from ‘A’ through ‘J’ and displayed on the graphical user interface in alphabetical order from left to right in a second single row;
each respective input for the individual's lower primary teeth are lettered from ‘K’ through ‘T’ and displayed on the graphical user interface in reverse alphabetical order from right to left in a third single row; and
each respective input for the individual's lower permanent teeth are numbered from eighteen through thirty one and displayed on the graphical user interface in reverse numerical order from right to left in a fourth single row.

5. The computer-implemented data processing method of claim 4, wherein the graphical user interface is configured to display the plurality of inputs corresponding to the plurality of teeth associated with the individual such that the first row is positioned above the second row, the second row is positioned above the third row, and the third row is positioned above the fourth row on the graphical user interface

6. The computer-implemented data processing method of claim 5, wherein the graphical user interface is configured to enable a user to chart dental information substantially without knowledge of technical dental or medical terms.

7. The computer-implemented data processing method of claim 1, further comprising:
providing a database comprising a plurality of predefined text or graphic entries, each of the plurality of predefined text or graphic entries being associated with one or more of the plurality of inputs corresponding to the plurality of teeth associated with the individual, the plurality of observation inputs, or the plurality of treatment inputs on a single display screen.

8. The computer-implemented data processing method of claim 7, wherein generating the summary report further comprises:
retrieving one or more of the plurality of predefined text or graphic entries associated with the at least one first input and the at least one second input; and
compiling the summary report by including the retrieved one or more of the plurality of predefined text or graphic entries associated with the at least one first input and the at least one second input.

9. The computer-implemented data processing method of claim 1, wherein processing the input data comprises modifying a data structure to include the at least one first input and the at least one second input.

10. The computer-implemented data processing method of claim 1, further comprising:
receiving a user selection of a snap shot link; and
at least partially in response to receiving the user selection of the snap shot link, providing a snap shot interface comprising a summary of patient information corresponding to the individual, the summary of patient information comprising:
a current operative treatment plan section comprising information corresponding to recommended treatments,
one or more referral sections comprising information corresponding to referred specialists or work performed by other specialists or practitioners,
a most recent examination section comprising information corresponding to examination or radiograph dates, and
an appointment section comprising information corresponding to appointments scheduled in the future.

11. The computer-implemented data processing method of claim 10, further comprising:
receiving a user selection of a health history summary link;
at least partially in response to receiving the user selection of the health history summary link, providing a health history summary interface comprising information corresponding to any allergy or other medical alert and responses to questions deemed medically relevant to treatment of the individual.

12. The computer-implemented data processing method of claim 1, further comprising a visual representation of at least one dental radiograph corresponding to the plurality of teeth associated with the individual.

13. A computer-readable storage medium storing computer-executable instructions for:
providing a graphical user interface for charting dental information for each of a plurality of teeth associated with the individual, the graphical user interface comprising:
a plurality of inputs corresponding to the plurality of teeth associated with the individual;
a plurality of observation inputs, each of the plurality of observation inputs being associated with a particular observation; and
a plurality of treatment inputs, each of the plurality of treatment inputs being associated with a particular treatment;
electronically receiving, input data comprising:
at least one first input of the plurality of inputs, the at least one first input comprising a selection of a particular tooth of the plurality of teeth; and
at least one second input associated with the at least one first input, the at least one second input comprising an input selected from a group consisting of:
at least one observation; and
at least one treatment;
providing a graphical representation on the graphical user interface of the particular tooth associated with the at least one first input, the graphical representation comprising a representation of the at least one observation or the at least one treatment corresponding to the at least one second input.

14. The computer-readable storage medium of claim 13, further comprising:
processing the input data by:
electronically associating the particular tooth with the at least one observation or the at least one treatment; and
digitally storing the input data associating the particular tooth with the at least one observation or the at least one treatment in an electronic record for the individual.

15. The computer-readable storage medium of claim 14, further comprising:
generating, based on the input data, a summary report of the at least one observation or the at least one treatment for the particular tooth, the summary report comprising:
a plain-language description of the at least one observation or the at least one treatment; and
a diagrammatic representation of the particular tooth relative to the plurality of teeth.

16. The computer-readable storage medium of claim 13, wherein the graphical user interface comprises a menu having a plurality of navigation links, the plurality of navigation links comprising:
a caries risk assessment link to a caries risk interface comprising a plurality of user-selectable assessment inputs and a plurality of user-selectable treatment recommendations;
an orthodontics tracking link to an ortho-tracking interface configured to accept user input corresponding to an orthodontics status of the individual;
a hygiene link to a hygiene interface comprising a plurality of user-selectable inputs corresponding to gingival health, oral hygiene, and oral hygiene instructions; and
an examination and treatment report link to an examination and treatment report interface comprising a summary report having a plain-language description of the at least one observation or the at least one treatment for the particular tooth.

17. A computer-implemented data processing method for electronically charting dental information for an individual, the method comprising:
accessing an electronic record for the individual;
providing a plurality of graphical user interfaces corresponding to the electronic record associated with the individual and one or more dental examinations or treatments associated with the individual,
receiving input via one or more of the plurality of graphical user interfaces; and
digitally updating the electronic record for the individual according to the input,
wherein a first graphical user interface of the plurality of graphical user interfaces comprises:
a plurality of inputs corresponding to the plurality of teeth associated with the individual,
a plurality of observation inputs, each of the plurality of observation inputs being associated with a particular observation, and
a plurality of treatment inputs, each of the plurality of treatment inputs being associated with a particular treatment.

18. The computer-implemented data processing method of claim 17, wherein the first graphical user interface further comprises:
at least one visual representation of at least one dental radiograph corresponding to the plurality of teeth associated with the individual;
a graphical representation on the graphical user interface of a tooth associated with a selection of an input of the plurality of inputs corresponding to the plurality of teeth, the graphical representation comprising a representation of at least one observation associated with a selection of an observation input of the plurality of observation inputs, or a representation of at least one treatment associated with a selection of a treatment input of the plurality of treatment inputs.

19. The computer-implemented data processing method of claim 17, wherein at least one input of the plurality of inputs corresponding to the plurality of teeth associated with the individual comprises an indicator visually indicating that there is an observation associated with the tooth, that a treatment is recommended for the tooth, or that a treatment has been completed for the tooth.

20. The computer-implemented data processing method of claim 17, wherein a second graphical user interface of the plurality of graphical user interfaces comprises:
a summary report of at least one observation or at least one treatment for a particular tooth, the summary report comprising:
a plain-language description of the at least one observation or the at least one treatment; and
a diagrammatic representation of the particular tooth relative to the plurality of teeth.
